(19) United States
(12) Patent Application Publication
(10) Pub. No.: US 2019/0183350 A1
(43) Pub. Date: Jun. 20, 2019

(54) MEASUREMENT SYSTEM AND METHOD FOR CHARACTERIZING TISSUE

(71) Applicant: OPUS NEOI GMBH, Zuerich (CH)

(72) Inventors: Mathias BONMARIN, Zuerich (CH); Patrick VON SCHULTHESS, Zuerich (CH); Simon FAHRNI, Zuerich (CH); Tobias GRUENDLER, Sirnach (CH); Pascal KUENZI, Uster (CH); Nils REINKE, Winterthur (CH)

(73) Assignee: OPUS NEOI GMBH, Zuerich (CH)

(21) Appl. No.: 16/307,981

(22) PCT Filed: Jun. 30, 2016
(86) PCT No.: PCT/CH2016/000099

§ 371 Date: Dec. 7, 2018

(51) Int. Cl. A61B 5/01

(52) U.S. Cl. CPC A61B 5/015; A61B 5/443

(57) ABSTRACT

A measurement system (1) for characterizing sample tissue (4; 4a, 4b) in a non-invasive way comprises a stimulation device (3; 3a, 3b; 3a, 3c) for thermal stimulation of the sample tissue (4; 4a, 4b), a temperature sensor (2; 2a, 2b) for capturing at least one temperature profile of the sample tissue (4) and a computing unit (11) connected to the temperature sensor (2; 2a, 2b) for processing data delivered by the temperature sensor (2; 2a, 2b). Thermal radiation radiated by the sample tissue (4; 4a, 4b) from at least a measurement area (5) is detectable as a result of the thermal stimulation by the temperature sensor (2; 2a, 2b) during the thermal stimulation of the sample tissue (4; 4a, 4b). The stimulation device comprises a temperature regulating body (3; 3a, 3b; 3a, 3c) which is in continuous contact with the sample tissue (4; 4a, 4b) in a contact area (6) during an entire measurement period. During a measurement period the temperature regulating body (3; 3a, 3b; 3a, 3c) has a different temperature than the sample tissue (4; 4a, 4b).

### BACKGROUND

The invention relates to a measurement system and a method for characterizing organic tissue according to the respective independent claim.

### PRIOR ART

Methods for characterizing tissue are known in several technological fields. For example, such methods are used in the characterization of skin tissue for detecting lesions. Most of these methods are based on a visual examination of the skin. This approach is subjective and after a visual characterization of the skin strongly depends on the experience of the respective physician.

A number of devices and examination methods have therefore been developed for simplifying this characterization and for enabling a better precision. The scientific basis of such devices is the active thermography.

The active thermography uses a heating or cooling of a tissue region and a subsequent recording of the surface temperature of the tissue which is susceptible of containing lesions. Some lesion types of skin have different thermo-physical properties than healthy skin. Particularly in case of cancer lesions, a higher metabolic heat development and a higher blood perfusion rate is expected.

Heating or cooling the tissue generates a non-stationary temperature gradient in the tissue, which in turn influences the distribution of surface temperature. If a region of the tissue has different thermo-physical properties as compared to its environment, particularly in a subjacent tissue layer, e.g. a different density, heat capacity, heat conductivity, etc., then this region influences the heat transfer and consequently also the time-dependent surface temperature of the tissue. By monitoring the time-dependent surface temperature, it is therefore possible to detect areas with different thermo-physical properties below the surface. In case of the active thermography, the tissue is heated by conduction, convection or absorption and cooled by conduction or convection. The energy supply can be continuous or periodical.

The principles of the active thermography can be found e.g. in the document “Infrared thermal imaging” by M. Vollmer and K. P. Mollmann, Wiley-VCH, 2010.

The article “Thermal transport characteristics of human measured in vivo using ultrathin conformal arrays of thermal sensors and actuators” by Webb, Pielak et. al., published Feb. 6, 2015, describes human clinical studies using mechanically soft arrays of thermal actuators and sensors that laminate onto the skin to provide rapid, quantitative in vivo determination of both the thermal conductivity and thermal diffusivity of skin in a non-invasive manner.

To summarize, visual characterization of tissue is subjective and unprecise. Some available systems use an initial stimulation and measure temperature development afterwards, which is also relatively unprecise. Other systems use a continuous or periodic stimulation and detect temperature variations of the tissue during the stimulation. These systems are precise; however, they are expensive due to the multitude of apparatuses and the complexity of synchronizing the stimulation unit with the data acquisition unit.

### DESCRIPTION OF THE INVENTION

It is the objective of the invention to reduce the disadvantages mentioned above.

This objective is reached in a first aspect of the invention by a measurement system for characterizing organic tissue. The measurement system for characterizing sample tissue in a non-invasive way comprises at least one stimulation device for thermal stimulation of the sample tissue, at least one temperature sensor for capturing at least one temperature profile of the sample tissue and a computing unit connected to the temperature sensor for processing data delivered by the temperature sensor. Thermal radiation radiated by the sample tissue from a measurement area as a result of the thermal stimulation is detectable by the at least one temperature sensor during the thermal stimulation of the sample tissue. The at least one stimulation device comprises at least one temperature regulating body which is in continuous contact with the sample tissue in a contact area during an entire measurement period. During a measurement period, the temperature regulating body has a different temperature than the sample tissue.

Furthermore, the objective is reached by a method for characterizing tissue. The method for characterizing sample tissue in a non-invasive way is carried out by means of the measurement system according to the first aspect of the invention. The method comprises the following steps:
setting a desired initial stimulation temperature of the at least one temperature regulating body, corresponding to a first contact of said temperature regulating body with the sample tissue, such that an initial temperature difference between said initial stimulation temperature and an initial tissue temperature is greater than zero,
positioning the at least one temperature regulating body onto the sample tissue such that it contacts the latter in the contact area, adjacent to the measurement area,
positioning the temperature sensor at a distance from the measurement area, wherein the distance is chosen such that substantially the entire measurement area can be monitored by the temperature sensor,
triggering capture of the temperature variations of the sample tissue by the temperature sensor, wherein capture is performed at predefined time intervals and transferring a corresponding captured temperature profile for each capture to the computing unit,
calculating at least one tissue characterizing parameter based on at least one of the captured temperature profiles by the computing unit and outputting calculation results.

The measurement system and the method according to the invention allows a precise and verifiable way of characterizing tissue, particularly identifying tissue regions differing from adjacent tissue regions. Furthermore, as the stimulating device is a temperature regulating body, the costs are reduced due to the simple construction of said temperature regulating body.

### SHORT DESCRIPTION OF THE DRAWINGS

In the following embodiments of the invention are described by means of the drawings. It is shown in:

FIG. 1 a first embodiment of a measurement system according to the invention;

FIG. 2 a second embodiment of a measurement system according to the invention;

FIG. 3 a third embodiment of a measurement system according to the invention;

FIG. 4 a fourth embodiment of a measurement system according to the invention;

FIG. 5 a fifth embodiment of a measurement system according to the invention;

FIG. 6 a sixth embodiment of a measurement system according to the invention; and

FIG. 7 a seventh embodiment of a measurement system according to the invention;

### WAYS OF CARRYING OUT THE INVENTION

The term “measurement area” is understood as an entire surface of the sample tissue, which faces one temperature sensor and is measured by the latter. The term shall be understood in this context as also encompassing measurements below the surface of the sample tissue.

The term “measurement system” is understood as a device or multiple devices coupled to one another. It may also imply data processing and presentation.

The term “characterizing” in connection with a sample or a parameter, as used herein, is understood as determination of one or more parameters indicative of a physical property of the sample. The determination implies measurements but may also encompass calculations based on measured values. In this context it is mentioned that a “characterizing parameter” determined by the method according to the invention may be used by a physician for diagnose purposes in case the sample is human or animal tissue (e.g. skin), as said physical property may indicate anomalies in the tissue structure.

The measurement device 1 can be used for characterizing sample tissues in vitro as well as in vivo.

It is understood that the measurement system may comprise fixing elements (not shown) if used for in vivo measurements. In other words, fixing elements for keeping the skin to be inspected immobile are provided, e.g. fixing elements for fixing an arm of a human subject to be inspected. These fixing elements may comprise alternatively or additionally elements for fixing the temperature regulating body 3 substantially on the same contact area, thus avoiding that it shifts on the skin. In this way erroneous measurement results are avoided.

In the context of the present document, the term “adjacent” is understood to encompass a side-by-side arrangement as well as a concentric arrangement of the related neighboring surfaces or areas.

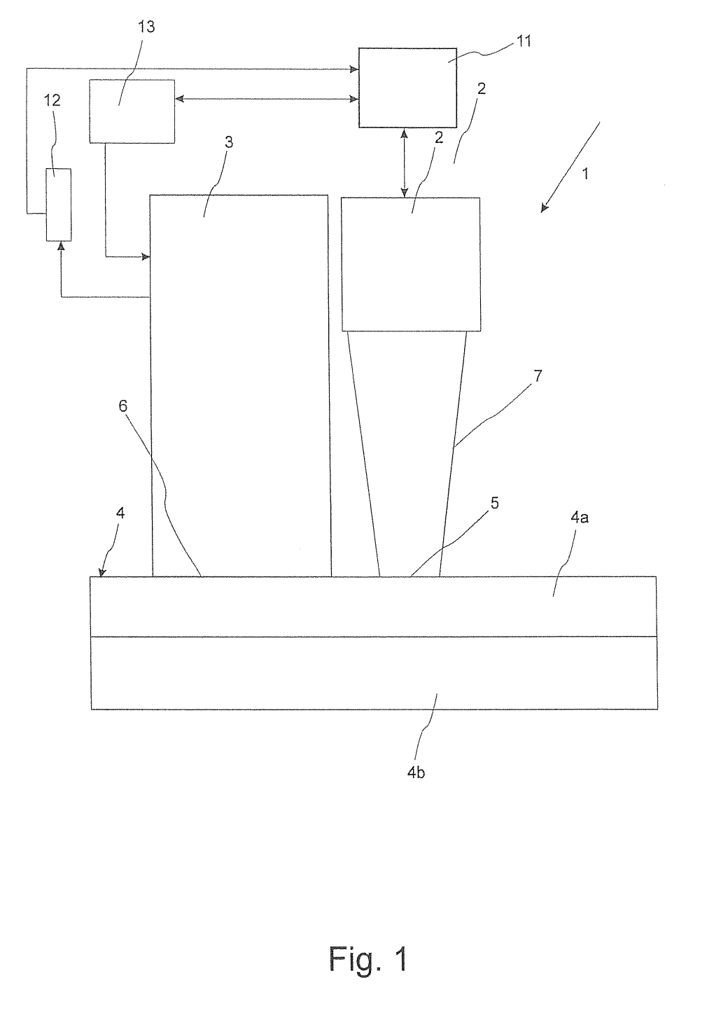

FIG. 1 shows a first embodiment of a measurement system 1 for characterizing sample tissue 4 in a non-invasive way according to the invention. The measurement system 1 comprises one stimulation device 3 for thermal stimulation of the sample tissue 4, one temperature sensor 2 for capturing at least one temperature profile of the sample tissue 4 and a computing unit 11 connected to the temperature sensor 2 for processing data delivered by the temperature sensor 2. The stimulation device is a temperature regulating body 3 positioned on the sample tissue 4, which is in continuous contact with the sample tissue 4 in a contact area 6 during an entire measurement period. The temperature sensor 2 covers a measurement area 5 on the sample tissue 4 surface, which is schematically and exemplarily illustrated in the figure by lines 7. Of course the extension of said measurement area may differ from the one shown in the figure.

During a measurement period the temperature regulating body 3 has a different temperature than the sample tissue 4. In this way it is made sure that thermal transfer between the temperature regulating body 3 and the sample tissue 4 is occurring during a measurement period.

The thermal radiation radiated by the sample tissue 4 from the measurement area 5 as a result of the thermal stimulation by the temperature regulating body 3 is detectable by the temperature sensor 2 during the thermal stimulation of the sample tissue 4. In other words, the measurement is carried out simultaneously with the stimulation and not after stimulation. In this way measurement results are more precise due to avoiding radiation loss which would occur in case of a sequential measurement process.

The temperature sensor 2 is preferably a contactless temperature sensor. In embodiments, the temperature sensor is a thermopile sensor or a thermographic camera. These types of sensors are known and are therefore not described here in more detail. Such devices may have a precision of 1/100 degree Celsius. One manufacturer for such sensors is the company MEAS Deutschland GmbH, Germany. The higher the precision of the thermopile sensor 2, the more accurate are the measurements. A higher precision also means that smaller temperature gradients between the stimulation device 3 and the sample tissue 4 can still yield good measurement results.

The temperature regulating body 3 and the temperature sensor 2 are arranged in such a way that the contact area 6 and the measurement area 5 are adjacent. Particularly, the contact area 6 doesn't surround the measurement area 5 or vice versa. By arranging the temperature regulating body 3 and the temperature sensor 2 in this way, it is made sure that the recorded temperature profile is influenced as little as possible by losses due to radiation of the sample tissue in areas which are not covered by the temperature sensor 2.

The measurement system 1 is adapted in such a way that it can be operated based on a cooling effect as well as on a heating effect, as desired by the user. Consequently, the temperature regulating body 3 may be a cooling body or a heating body, depending on what type of sample tissue shall be characterized. In one main application of the measurement system 1 which aims at characterizing skin as sample tissue, it is preferred that the temperature regulating body is a cooling body due to the reasons set forth. The measurement system 1 is simple, as a natural temperature gradient between the temperature regulating body 3 and the tissue exists, that is: skin temperature at the skin surface is typically around 33° C. or 34° C. and ambient temperature in a measurement environment is normally room temperature, e.g. 20° C. Hence, if the temperature regulating body has been exposed to the ambient temperature for long enough in order to stabilize its temperature to the ambient temperature, e.g. 20° C., it no additional steps are needed for creating the required temperature gradient between itself and the skin, which is necessary for the measurement. Therefore, not only the measurement setup is significantly simplified but also costs for additional devices like coolers are saved. Another reason is that this option doesn't bring the risk that the temperature regulating body burns the skin if the measurement is carried out in vivo.

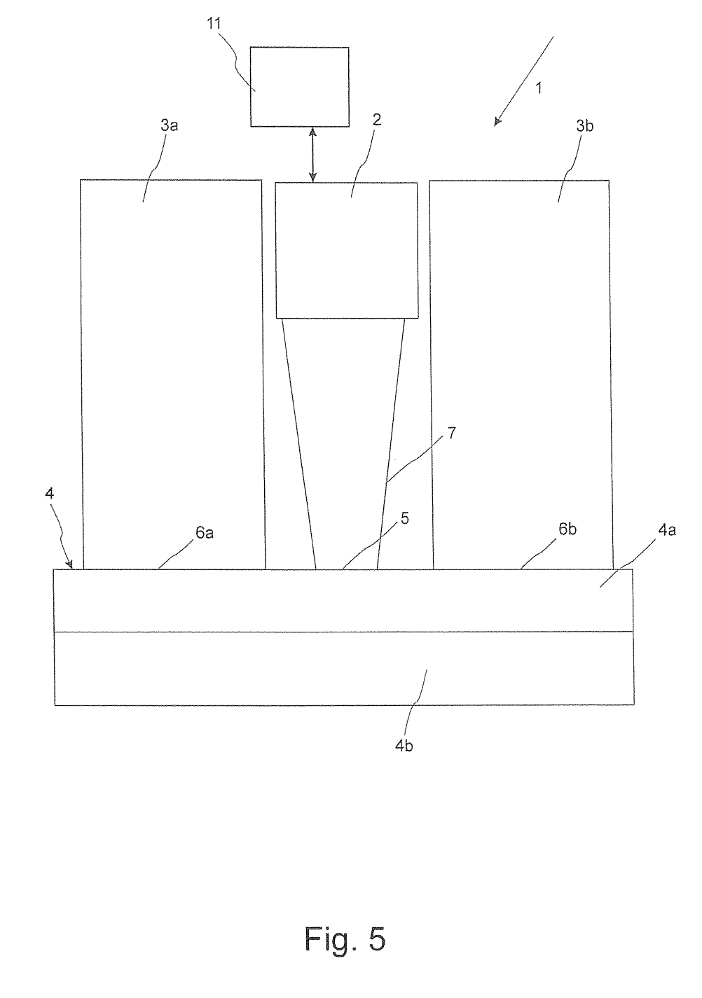

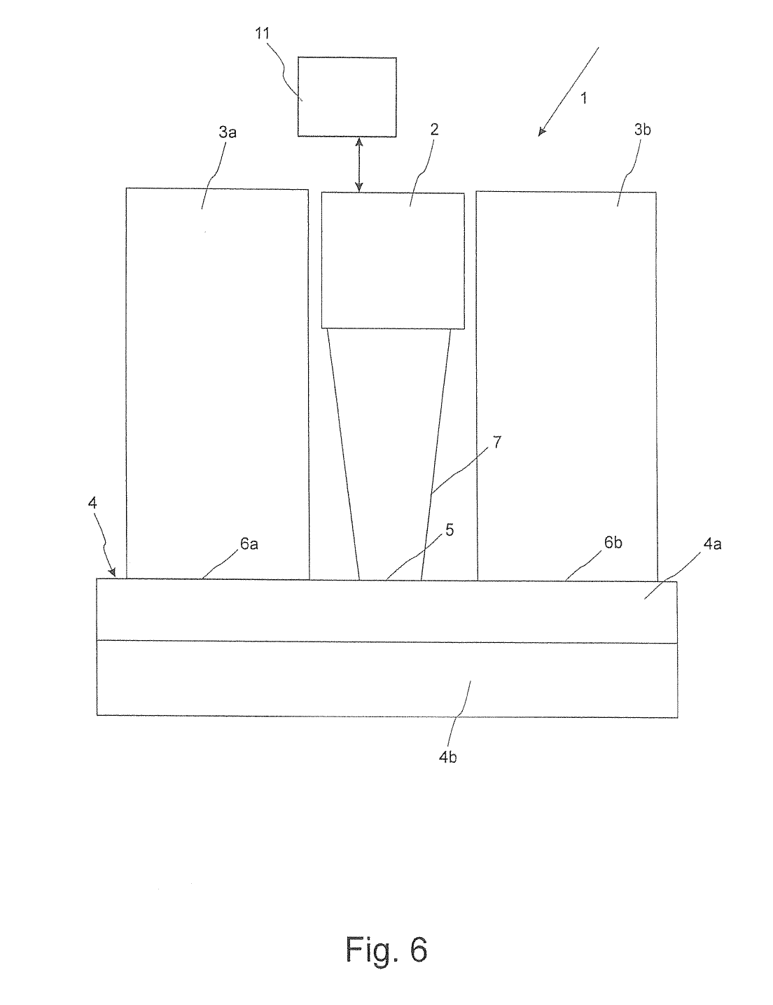

The measurement system also comprises an optional auxiliary temperature sensor 12 provided for monitoring a temperature of the temperature regulating body 3. If more than one temperature regulating body is used (e.g. FIG. 5 or 6), it is optionally possible to provide an auxiliary temperature sensor 12 for each one of the temperature regulating bodies 3a, 3b, 3c. The auxiliary temperature sensor or sensors are connected to the computing unit 11 for feeding temperature data readings from the temperature regulating body 3. In this way it is advantageously possible to monitor temperature changes of said regulating body 3. For example, it is possible to define a trigger temperature of the temperature regulating body or a trigger temperature gradient between the temperature regulating body and the sample tissue, the sample tissue temperature being determined by the temperature sensor 2. This trigger temperature or temperature gradient may be used for determining when to terminate a measurement process. For this task, the temperature gradient mentioned above is most preferred, as it provides information about the interaction between the temperature regulating body 3 and the sample tissue 4. If the temperature gradient is too low, this is indicative of a reduced temperature transfer. In this case the user can contemplate to end the measurement. In another case, if the temperature gradient substantially equals zero, the measurement may also be terminated automatically, as no further temperature changes in the sample tissue 4 are expected to occur.

For the purposes of this document the stimulation device 3 is considered a passive regulating element as it has no means for changing its own temperature. However, it is also possible to alternatively or additionally use an active temperature regulation. In case the stimulation device 3 is based on an active temperature regulation of the temperature regulating body 3, thus also comprises means 13 for cooling down or heating up the temperature regulating body 3, the auxiliary temperature sensor 12 may also be used to provide the temperature data required to control the means for cooling down or heating up the temperature regulating body 3.

Means 13 for cooling down or heating up the temperature regulating body 3 are known to the skilled person and will not be described in detail herein. It is noted that such means can also be used in conjunction with the subsequent embodiments. Particularly, more than one such means 13 may be provided, e.g. in the embodiments according to FIGS. 5 and 6 one for each temperature regulating body 3a, 3b, 3c.

In the following, other embodiments of the measurement system according to the invention are described. For simplicity reasons, only the differences to previously described embodiments are explained.

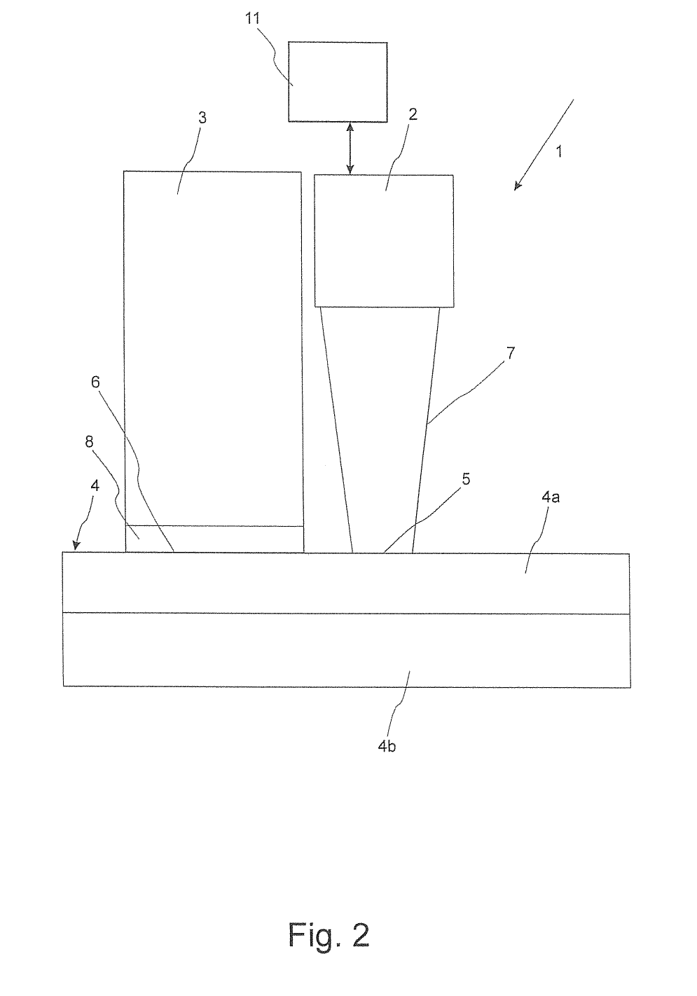

FIG. 2 shows a second embodiment of a measurement system according to the invention. This embodiment differs from the embodiment according to FIG. 1 in that the temperature regulating body comprises a layer or a coating 8 adapted to increase heat transfer between the sample tissue 4 and the temperature regulating body 3. Certainly, in case multiple temperature regulating bodies are used (see description of FIG. 5 or 6) such a coating or layer may be applied to both temperature regulating bodies or only to one of them. Such a coating 8 may be a heat conductive silicon layer having a thickness of e.g. between 100 and 10000 μm. The purpose of this coating or layer 8 is to enhance heat transfer by adjusting to the contact area surface of the sample tissue and filling up potential air gaps in the contact area 6, which would otherwise act as insulation and prevent a good heat transfer between the temperature regulating body 3 and the sample tissue 4. Therefore, a more efficient heat transfer is reached.

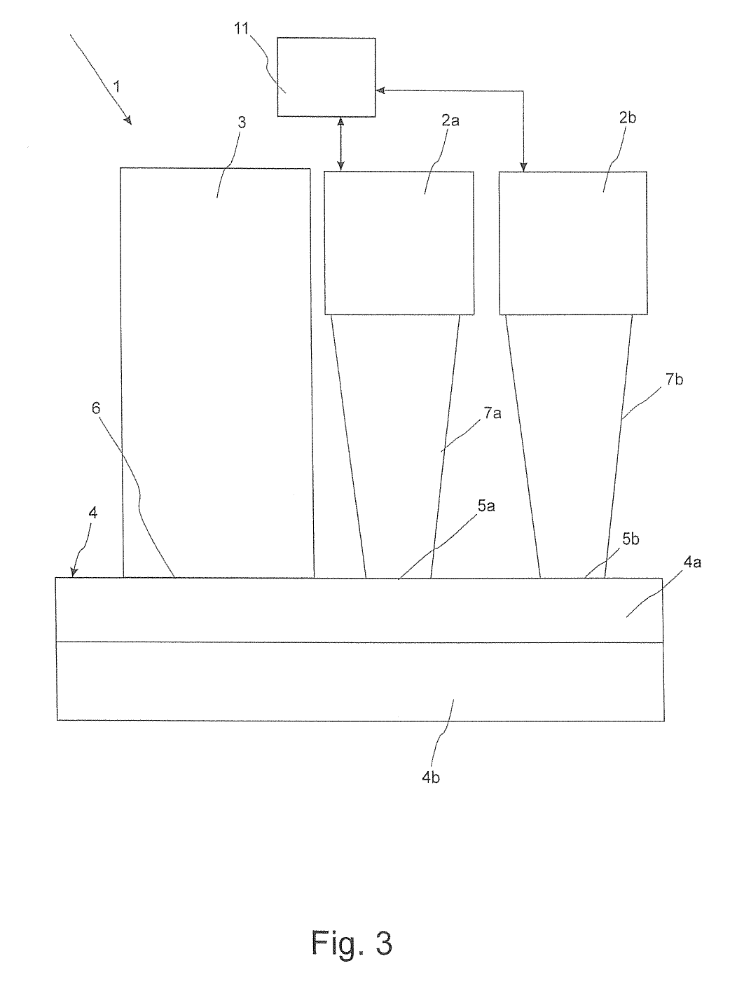

FIG. 3 shows a third embodiment of a measurement system 1 according to the invention. This embodiment differs from the ones already described in that two temperature sensors 2a, 2b are provided for measuring temperatures of adjacent volumes or areas or of different points of the sample tissue 4. This type of setup may be advantageous if a large sample tissue shall be measured, which cannot be covered by a single temperature sensor. In this context it is noted that an array of temperature sensors may alternatively be used, comprising a plurally of sensor units pooled in one single sensor device.

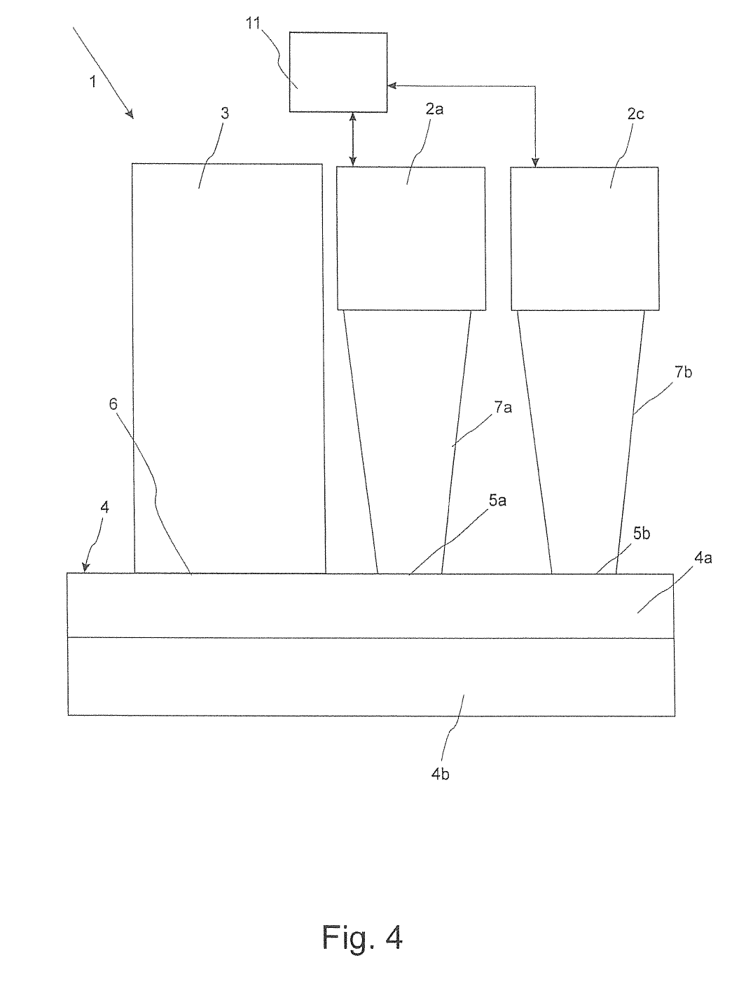

FIG. 4 shows a fourth embodiment of a measurement system 1 according to the invention. This embodiment differs from the one of FIG. 3 only in that the temperature sensor 2b of FIG. 3 is replaced by a sensor of electromagnetic radiation 2c. This embodiment is advantageous for detection in a different wavelength spectrum than the wavelength of the temperature sensor 2b in order to gain parameters of different depths of the tissue by for example measuring the reflectivity of the surface of the measurement area. The penetration of infrared light into the tissue is highly dependent on the wavelength of the infrared light used.

FIG. 5 shows a fifth embodiment of a measurement system 1 according to the invention. This embodiment differs from the already described embodiments in that two temperature regulating bodies 3a, 3b are provided, which contact the sample tissue 4 such that the measurement area 5 of the temperature sensor 2 is located between the two temperature regulating bodies 3a, 3b. Accordingly, two contact areas 6a, 6b are provided, each one being attributed to one of the temperature regulating bodies 3a, 3b. Preferably, the two temperature regulating bodies have substantially the same initial temperature when a measurement is started. This setup advantageously allows a same temperature influence on the sample tissue 4 from two sides of the measurement area 5, such that a more uniform temperature distribution on the entire surface of the measurement area 5 is reached. In contrast to this, usage of a single temperature regulating body 3 may lead to a non-zero measurement area temperature gradient between the temperature of the measurement area 5 in the vicinity of the temperature regulating body and the temperature of the measurement area 5 on its far side with respect to the location of the temperature regulating body. Of course, such an embodiment also yields good results but there is more computational effort required for taking into account said measurement area temperature gradient.

FIG. 6 shows a sixth embodiment of a measurement system 1 according to the invention. Just like the fifth embodiment, in this case there are also two temperature regulating bodies 3a, 3b provided on the sample tissue 4, which contact the sample tissue 4 such that the measurement area of the temperature sensor 2 is located between the two temperature regulating bodies 3a, 3b. Even though this embodiment is structurally identical to the embodiment of FIG. 5, it is mentioned here as independent embodiment due to its additional or alternative measurement type. This measurement type relates to measuring thermal conductivity between the two contact areas 6a, 6b. In this case it may be preferred that the two temperature regulating bodies have different initial temperatures at the beginning of a measurement session; e.g. one of the bodies could act as a heater and the other one as a cooler, or both may act in the same way but with different initial temperatures, wherein these initial temperatures are both lower or higher than the initial sample tissue temperature. This measurement type may advantageously be used to detect if tissue portions acting as heat transfer barriers exist inside the skin, hinting that a tissue irregularity may be present. It is furthermore possible to derive a degree of humidity of the skin from the speed of thermal transfer across the measurement area 5 by taking into account, during characterization, the typical transfer speed of thermal radiation in water.

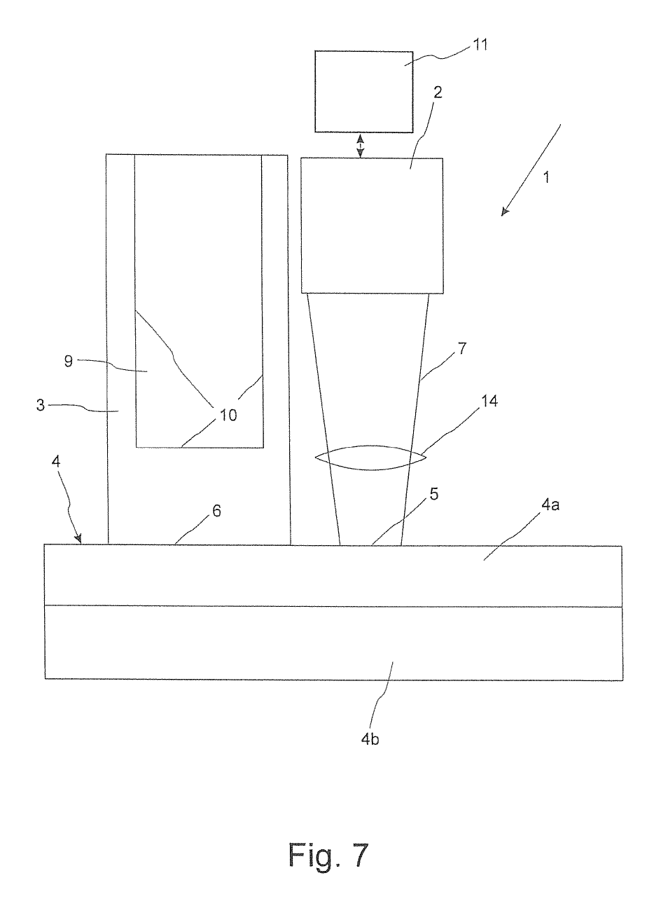

FIG. 7 shows a seventh embodiment of a measurement system according to the invention. This embodiment differs from the embodiment of FIG. 1 in that it comprises a lens 14 and a reservoir body 9. It is noted that said new elements are independent from one another and may also be used in the other aforementioned embodiments; they are not shown in the figures of said embodiments for simplicity reasons.

In accordance with the aforementioned two types of temperature regulating bodies (heater, cooler) in this embodiment there are also two possible configurations. In the first case, the temperature regulating body 3 is a cooling body adapted to be cooled by a cold reservoir body 9 having a higher heat capacity than the cooling body 3. The cold reservoir body 9 is in contact with the cooling body 3. It also may be integrated in the cooling body 3, as suggested by the figure. In the second case, the temperature regulating body 3 is a heating body adapted to be heated by a heat reservoir body 9 having a higher heat capacity than the heating body 3 and which is in contact with the heating body 3. It also may be integrated in the heating body 3. Advantageously, by using a heat/cold reservoir, it is possible to keep the temperature difference between the temperature regulating body and the sample tissue high enough to measure several times in a row and to keep the temperature of the body stable, in other words less influenced by the ambient temperature.

In both cases there is a non-zero reservoir temperature gradient between the initial reservoir body temperature and the initial temperature regulating body temperature. In both cases the reservoir body has auxiliary contact surfaces 10 with the cooling body or the heating body, respectively, where the heat transfer between the two bodies takes place. Of course, the principle of enhancing heat transfer by applying a layer or coating, as described in connection with FIG. 2, also applies for this reservoir contact surfaces or surface 10. The shape of the reservoir body is preferably chosen such that a maximum reservoir contact surface 10 within the given dimensions of the temperature regulating body 3 is reached.

The reservoir body 9 as depicted in the figure is considered to be passive, i.e. it doesn't comprise means for altering its temperature during a measurement. However, an active temperature regulation of the reservoir body by the means 13 according to FIG. 1 may also be used.

The reservoir body is preferably removable from the temperature regulating body, e.g. such that it can easily be introduced into a refrigerator.

Furthermore, a lens 14 attributed to the temperature sensor 2 is arranged between said temperature sensor 2 and said measurement area 5. Alternatively or additionally a filter may be arranged in the measurement path 7 between the temperature sensor 2 and the measurement area 5.

The lens may be used to amplify the signal from the sample tissue on the temperature sensor by concentrating the temperature radiation rays to the temperature sensor.

The filter may be used to filter out radiation at wavelengths which are not interesting for the measurement and which might potentially produce erroneous measurement results. The filter is used to detect certain parameters of the tissue layers, that is: the depth of penetration of the sample tissue varies depending on the wavelength of the radiation. With a filter, certain signals can be separated.

In the following certain aspects of the temperature regulating body as well as alternative embodiments are described.

The temperature regulating body may for example be an aluminium or steel body in all embodiments of the invention. Other materials satisfying the aforementioned requirements to the material may alternatively be used.

In the already described embodiments of FIGS. 1 to 7 the temperature regulating body is assumed to be a block or a hollow body arranged on the side of the temperature sensor or sensors. However, it may be preferred that the temperature regulating body is a tube which is open on its both front faces, particularly a cylindrical tube, with a substantially central opening around the axis of the tube. For simplicity reasons it is again referred to some of the figures, however with a different interpretation, for describing this embodiment of the temperature regulating body, the figures being FIGS. 1 to 4 and FIG. 7. According to this embodiment, the drawn temperature regulating body in each of said figures is interpreted as a section view on one side through the wall of the tube. Therefore the figures are interpreted in such a way that the temperature regulating body surrounds the temperature sensor or sensors. Consequently, the contact area and the measurement area are also adjacent, but the contact area 6 surrounds the measurement area 5 at least partially.

Which type of temperature regulating body is chosen depends on the setup and the environment. The arrangement side by side with the temperature sensor has the advantage that the environmental temperature, which is assumed to be substantially constant, is also the temperature of the air column between the temperature sensor and the sample tissue, such that its influence on measurements can be neglected. Contrary to this, when the temperature regulating body surround the temperature sensor, a “micro climate” is created inside the tube, which also varies with the changing temperature of the temperature regulating body. On the one hand the tube-arrangement is suitable for cooling/heating the measurement area 6 in a uniform way from all sides. This allows a more precise characterization of the measurement area. On the other hand the arrangement with two temperature regulating bodies is suitable for measuring the temperature profile of a larger measurement area and, as the case may be, for determining spatial changes of the temperature distribution e.g. due to lesions, as mentioned at the beginning.

Thus, the temperature gradient of the sample tissue varies depending on the used setup.

In the following, the method for characterizing sample tissue in a non-invasive way by means of the measurement system described above is described. The method comprises the steps set forth below.

In a first step, a desired initial stimulation temperature of the temperature regulating body 3 is set. This initial temperature is the temperature of the stimulation device 3 at its first contact with the sample tissue, and it is chosen such that an initial temperature difference between said initial stimulation temperature and an initial tissue temperature is greater than zero. An advantage of using a simple temperature regulating body 3 becomes apparent in this context: it is very easy to set this initial temperature by simply placing the temperature regulating body 3 in a refrigerator, if necessary, or leaving it for a sufficient time at ambient temperature (room temperature), in case the temperature regulating body is a cooling body, or by simply placing the temperature regulating body 3 in an oven in case the temperature regulating body is a heating body.

In a second step, the stimulation device is positioned on the sample tissue such that it contacts the latter in the contact area 6, adjacent to the measurement area 5. This step may include also fastening the stimulation device on the sample tissue by means of the fixing elements mentioned at the beginning.

In a third step, the temperature sensor 2 is positioned at a distance from the measurement area 5. The distance is chosen such that substantially the entire measurement area 5 can be monitored by the temperature sensor 2. This step may also imply positioning a second temperature sensor in the same way (see FIG. 3, 4).

In a fourth step, capture of the temperature variations of the sample tissue by the temperature sensor is triggered by the computing device 11. Capture is performed at predefined time intervals. A sub-step consists in transferring a corresponding captured temperature profile for each capture to the computing unit 11.

In a fifth step, at least one tissue characterizing parameter is calculated based on at least one of the captured temperature profiles by the computing unit 11. Finally, calculation results are outputted in a suitable way for interpretation by the user. The tissue characterizing parameter or parameters are preferably chosen to be one of or a combination of: a heat capacity of the sample tissue, a thermal conductivity of the sample tissue, a tissue density, a spatial location of an abrupt change in a temperature distribution of the measurement area or a tissue layer.

In other words, first a measurement head comprising the temperature regulating body and the temperature sensor are placed on the tissue, thereby triggering temperature stimulation of the same. This results in lateral heat transport between the temperature regulating body and the sample tissue. Preferably, the initial stimulation temperature of the temperature regulating body 3 is chosen in such a way that the initial temperature difference between it and the initial tissue temperature is greater than a temperature resolution of the temperature sensor. For example, in case a thermopile sensor with the above mentioned resolution of 1/100 degree Celsius is chosen, the initial temperature difference is chosen to be greater than 1/100 degree Celsius, preferably at least 10 times greater than said temperature resolution of the temperature sensor. It is preferred that the initial temperature difference is of at least 0.5° C.

Subsequently, the time-dependent temperature course is measured for the measurement area 5 and finally the characterizing, physical parameter is extracted by solving mathematical equations or algorithms.

For the embodiments in which the temperature regulating body surrounds the temperature sensor or sensors the method is the same. The only difference is the initial placement of the temperature sensor and the temperature regulating body.

Preferably, the characterizing parameter is calculated by the computing unit by solving the bio-heat or Pennes equation:

$$ρ c \frac{\partial }{\partial t} T ( r , z , t ) + {ρ}_{b} {c}_{b} {ω}_{b} [ T ( r , z , t ) - {T}_{b} ] + Q = k {∇}^{2} T ( r , z , t )$$

wherein ρ is the density of the sample tissue, c is a constant modelling the heat storage capacity of the sample tissue, T is the temperature of the sample tissue at the location r, z at the time instant t and k is the temperature conductivity of the sample tissue. ρ_{b} is the blood density, c_{b} is a constant modeling the heat storage capacity of blood, ω_{b} is the tissue perfusion, T_{b} is the blood temperature, and Q is the tissue metabolic heat. It is mentioned that the above equation is expressed in cylindrical coordinates r, z for simplicity reasons. Then, Laplace- and Hankel-transforms can be used to solve the above-mentioned equation with appropriate boundary conditions.

The capture of the temperature variations is ended either if a predefined time span has lapsed or if a current temperature difference between a current stimulation temperature and a current tissue temperature has reached a predefined threshold temperature difference. As mentioned, the latter criterion may e.g. be monitored by using Lhe auxiliary temperature sensor 12 mentioned in connection with FIG. 1.

In preferred embodiments of the method, reference temperature profiles may be recorded in advance using a reference sample tissue of the type of the sample tissue to be characterized, known to be homogenous. This measure may facilitate characterization by comparing the reference temperature profiles with the measured temperature profiles.

The sample tissue may comprise multiple layers, which is e.g. typical for skin. The present solution makes it possible to characterize the sample tissue of deeper layers and not only the top layer or the surface of the sample tissue. This is done by identifying borders between layers. As each layer typically has different physical properties, like heat conductivity, the heat transfer between the stimulation device and the tissue is different in the different layers, thereby leading to a possibility of recognizing the respective layer by comparing temperature profiles on either side of the border between two layers. If the sample tissue comprises more than one layer, a thickness and/or a water content of at least one of the layers may be calculated. Consequently, the measurement device according to the invention is preferably used for characterizing one or multiple layers of skin, particularly a surface layer and/or a subjacent layer of the surface layer. Advantageously, an ageing degree of the skin can be determined by measuring skin water content and thickness of the epidermis as surface layer of the skin by deriving these parameters from the parameters retrieved according to the above equation.

In embodiments having two temperature regulating bodies, the thermal conductivity of the sample tissue is determined by adjusting the initial stimulation temperatures of the temperature regulating bodies in such a way that a value of the initial tissue temperature is between the two initial stimulation temperatures of the temperature regulating bodies, preferably in the middle of the two initial stimulation temperatures.

The present invention has a number of advantages over known solutions: measurements are precise and yield objective results. By using a simple stimulation body complexity is reduced substantially. Usage of a continuous or periodic stimulation and detection of temperature variations of the tissue during the stimulation increases precision of the measurement.

While presently preferred embodiments of the invention are shown and described in this document, it is distinctly understood that the invention is not limited thereto but may be embodied and practiced in other ways within the scope of the following claims. Therefore, terms like “preferred” or “in particular” or “particularly” or “advantageously”, etc. signify optional and exemplary embodiments only.

1. Measurement system for characterizing sample tissue in a non-invasive way, comprising:
at least one stimulation device adapted to thermally stimulate the sample tissue,
at least one temperature sensor adapted to capture at least one temperature profile of the sample tissue,
a computing unit connected to the temperature sensor adapted to process data delivered by the temperature sensor,
wherein thermal radiation radiated by the sample tissue from at least a measurement area as a result of the thermal stimulation is detectable by the at least one temperature sensor during the thermal stimulation of the sample tissue,
wherein the at least one stimulation device comprises at least one temperature regulating body which is in continuous contact with the sample tissue in a contact area during an entire measurement period, wherein during a measurement period the temperature regulating body has a different temperature than the sample tissue.

2. The measurement system according to claim 1, wherein the at least one temperature sensor is a contactless temperature sensor, wherein optionally two temperature sensors are provided for
measuring temperatures of adjacent volumes or areas or of different points of the sample tissue.

3. The measurement system according to claim 1, wherein a lens and/or a filter attributed to each temperature sensor is arranged between said at least one temperature sensor and said at least one measurement area.

4. The measurement system according to claim 1, wherein an auxiliary temperature sensor is provided for monitoring a temperature of the temperature regulating body.

5. The measurement system according to claim 1, wherein the temperature regulating body and the at least one temperature sensor are arranged in such a way that the contact area and the measurement area are adjacent.

6. The measurement system according to claim 1, wherein, in case one temperature
regulating body is used, the temperature regulating body and the at least one temperature sensor are arranged in such a way that the contact area and the measurement area are adjacent, particularly wherein the contact area optionally surrounds the measurement area at least partially.

7. The measurement system according to claim 1, wherein two temperature regulating bodies are provided, which contact the sample tissue such that the measurement area of the at least one temperature sensor is located between the two temperature regulating bodies.

8. The measurement system according to claim 1, wherein the temperature regulating body or at least one of the temperature regulating bodies comprises or comprise, respectively, a layer or a coating adapted for increasing heat transfer between the sample tissue and the temperature regulating body or the at least one of the temperature regulating bodies, particularly wherein the layer or the coating is made of heat conductive silicon, wherein a thickness of the layer or coating is optionally between 100 and 10000 μm.

9. The measurement system according to claim 1, wherein the temperature regulating body is a cooling body adapted to be cooled by a cold reservoir body having a higher heat capacity than the cooling body and which is in contact with the cooling body or integrated in the cooling body or wherein the temperature regulating body is a cooling body adapted to be actively cooled by a cooling device.

10. The measurement system according to claim 1, wherein the temperature regulating body is a heating body adapted to be heated by a heat reservoir body having a higher heat capacity than the heating body and which is in contact with the heating body or integrated in the heating body or wherein the temperature regulating body is a heating body adapted to be actively heated by a heater.

11. The measurement system according to claim 1, comprising one temperature sensor for capturing at least one temperature profile of the sample tissue and a sensor of electromagnetic radiation for capturing in a different wavelength spectrum than the a wavelength covered by the temperature sensor for gaining parameters of different depths of the tissue or for measuring reflectivity of the surface of the measurement area.

12. Method for characterizing sample tissue in a non-invasive way by means of the measurement system according to claim 1, comprising:
setting a desired initial stimulation temperature of the at least one temperature regulating body, corresponding to a first contact of said temperature regulating body with the sample tissue, such that an initial temperature difference between said initial stimulation temperature and an initial tissue temperature is greater than zero,
positioning the at least one temperature regulating body on the sample tissue such that it contacts the latter in the contact area, adjacent to the measurement area,
positioning the temperature sensor at a distance from the measurement area, wherein the distance is chosen such that substantially the entire measurement area can be monitored by the temperature sensor,
triggering capture of the temperature variations of the sample tissue by the temperature sensor, wherein capture is performed at predefined time intervals and transferring a corresponding captured temperature profile for each capture to the computing unit,
calculating at least one tissue characterizing parameter based on at least one of the captured temperature profiles by the computing unit and outputting calculation results.

13. The method according to claim 12, wherein the capture of the temperature variations is ended either if a predefined time span has lapsed or if a current temperature difference between a current stimulation temperature and a current tissue temperature has reached a predefined threshold temperature difference.

14. The method according to claims 10, wherein the initial stimulation temperature of the at least one temperature regulating body is chosen in such a way that the initial temperature difference between it and the initial tissue temperature is greater than a temperature resolution of the temperature sensor, particularly wherein the initial stimulation temperature is chosen in such a way that said initial temperature difference is at least 10 times greater than said temperature resolution of the temperature sensor, wherein the initial temperature difference is optionally of at least 0.5° C.

15. The method according to claims 10, wherein the tissue characterizing parameter or parameters are chosen to be one of or a combination of: a heat capacity of the sample tissue, a thermal conductivity of the sample tissue, a tissue density, a spatial location of an abrupt change in a temperature distribution of the measurement area or a tissue layer.

16. The method according to claim 15, wherein, in case two temperature regulating bodies are used, the thermal conductivity of the sample tissue is determined by adjusting the initial stimulation temperatures of the temperature regulating bodies in such a way that a value of the initial tissue temperature is between the two initial stimulation temperatures of the temperature regulating bodies, particularly in the middle of the two initial stimulation temperatures.

17. The method according to claims 10, wherein the characterizing parameter is calculate by the computing unit by solving the heat equation, particularly wherein a thickness and/or a water content of at least one of the layers of the sample tissue is calculated if the sample tissue comprises more than one layer.

18. Method for characterizing one or multiple layers of skin, particularly a surface layer and/or a subjacent layer of the surface layer, comprising: providing the measurement device of claim 1, wherein an ageing degree of the skin is determined by measuring skin water content and thickness of the epidermis as surface layer of the skin by deriving it from the at least one tissue characterizing.

19. The measurement system according to claim 2, wherein the contactless temperature sensor is a thermopile sensor or a thermographic camera.

20. The measurement system according to claim 5, wherein the contact area doesn't surround the measurement area or vice versa.
